(19) United States
(12) Patent Application Publication
(10) Pub. No.: US 2021/0327591 A1
(43) Pub. Date: Oct. 21, 2021

(54) System for Efficiently Estimating and Improving Wellbeing

(71) Applicant: Koa Health B.V., Barcelona (ES)

(72) Inventors: Oliver Harrison, London (GB); Aleksandar Matic, Lloret de Mar (ES)

(21) Appl. No.: 17/359,488

(22) Filed: Jun. 25, 2021

Related U.S. Application Data
(63) Continuation-in-part of application No. PCT/EP2019/087036, filed on Dec. 26, 2019.

(30) Foreign Application Priority Data
Dec. 27, 2018 (EP) 18382995.1
Dec. 26, 2019 (EP) PCT/EP2019/087036

(51) Int. Cl. G16H 50/30; G16H 10/20; G16H 40/67; A61B 5/00; A61B 5/01; A61B 5/024; A61B 5/11; G06N 3/04

(52) U.S. Cl. CPC G16H 50/30; G16H 10/20; G16H 40/67; A61B 5/7475; A61B 5/6898; A61B 5/01; A61B 2560/0252; A61B 5/1118; A61B 5/4806; A61B 5/6801; A61B 5/0004; G06N 3/0472; A61B 5/02405

(57) ABSTRACT

A system for improving a user's wellbeing uses machine learning to evaluate statistical parameters and to recommend parameter ranges that will improve the user's wellbeing. The system receives user data including ambient and environmental data related to the user's location, physiological data related to the user's body, and behavioral data of the user. Statistical parameters are generated that characterize the data based on a time period corresponding to the data. A wellbeing score for the user is generated by applying a probabilistic model to the statistical parameters. The model determines a recommended range of values for the parameters related to the ambient data, environmental data, physiological data and behavioral data that will improve the user's wellbeing score based on the statistical parameters and the wellbeing score. Behavior modifications based on the recommended range of values for parameters are recommended to the user so as to improve the user's wellbeing score.

### CROSS REFERENCE TO RELATED APPLICATION

This application is filed under 35 U.S.C. § 111(a) and is based on and hereby claims priority under 35 U.S.C. § 120 and § 365(c) from International Application No. PCT/EP2019/087036, filed on Dec. 26, 2019, and published as WO 2020/136215 A1 on Jul. 2, 2020, which in turn claims priority from European Application No. EP18382995.1, filed in the European Patent Office on Dec. 27, 2018. This application is a continuation-in-part of International Application No. PCT/EP2019/087036, which is a continuation of European Application No. EP18382995.1. International Application No. PCT/EP2019/087036 is pending as of the filing date of this application, and the United States is an elected state in International Application No. PCT/EP2019/087036. This application claims the benefit under 35 U.S.C. § 119 from European Application No. EP18382995.1. The disclosure of each of the foregoing documents is incorporated herein by reference.

### TECHNICAL FIELD

The present invention has its application within the telecommunications sector and, more specifically, relates to the deployment of tools using electronic devices and communication electronic devices (e.g., mobile user terminals such as smartphones, tablets, wearable electronic devices, computers, etc.) that measure an send user's parameters and/or interact with the user, to estimate and improve his/her wellbeing. More particularly, the present invention relates to a method and electronic system for an efficient measurement, modeling and estimation of a user's wellbeing, which allows accurate recommendations to be generated that improve the user's wellbeing.

### BACKGROUND

Since 2008, the leading causes of death in the United States have resulted from personal choices (smoking, bad diet habits, drinking alcohol, etc.), and the same trend is seen in many countries worldwide.

In this context, behavioral change programs that address such personal choices have the potential to deliver considerable benefits to both the general health of the population and to the cost burden of disease across the population. However, for many people changing health-related behavior is very challenging. Oftentimes individuals are instructed by their doctors to keep certain psychological and/or physiological parameters (heart rate, blood pressure, blood sugar level, respiratory rate, respiratory depth, stress, etc.) within certain boundaries. But it is typically left to the individual to figure out the triggers that result in their non-healthy physiological parameters.

Another reason for the limited impact of behavioral change programs to date is that advice from healthcare professionals and public health campaigns is not personalized for each user to maximize its effectiveness.

Studies show that wellbeing improvement efforts are more effective when they are personalized to the unique environment, characteristics and behavior of the user. Technological advancements have brought many methods for profiling individuals according to pre-defined criteria, and for automatically detecting a user's activities, behaviors and states. These advancements have been exploited by a host of services that typically provide targeted content to users (i.e., advertising) aiming to persuade them to purchase a product or service. However, there are only a few that target a specific user's wellbeing. These approaches either rely on the user's segment or on a set of activities undertaken by the user in order to suggest beneficial actions towards improving mood or subjective wellbeing.

Furthermore, changing health-related behavior for long-term wellbeing benefits is very challenging as people typically perceive that the behavioral change will come with investing effort (such as doing sports) and/or sacrificing immediate pleasure (such as refraining from alcohol, tobacco, specific food, etc.) in short-term. For that reason it is beneficial for behavioral change programs to consider the perception of one's happiness and wellbeing. Any behavioral intervention will be more successful if it aims to maintain one's happiness, for which monitoring wellbeing becomes crucial.

The wellbeing of a person, also known as the subjective wellbeing (SWB), is a well-known term defined as the degree to which people have positive thoughts and feelings about their lives. In other words, subjective wellbeing represents a self-evaluation of an individual's life or experience. It includes reflective cognitive evaluations, such as life satisfaction and work satisfaction, interest and engagement, and effective (momentary) reactions to life events, such as joy and sadness.

The measurement or estimation of SWB is often based on self-reports. Two components of SWB have been extensively covered by the wellbeing literature:

1) reflective wellbeing (“life-satisfaction”), that is a semi-static component, and 2) experiential wellbeing (how a person feels in the moment), which is a dynamic component.

Both of these components have two orthogonal dimensions to be measured (and inferred through a SWB model):

Hedonic wellbeing (a user's subjective sense of pleasure)

Eudemonic wellbeing (a user's subjective sense of purpose)

Wellbeing is a self-evaluation and a subjective measure. Therefore it is challenging to infer wellbeing in a passive way. On the other hand, in order to develop engaging wellbeing recommender services, it is important to enable passive ways accurately to approximate the dynamics of SWB in order to decrease the burden of frequently asking users to evaluate their lives (life-satisfaction) and how they feel in the moment (experiential wellbeing). Asking users these questions frequently is also considered to be an intervention with an unpredictable outcome for different kinds of users. Moreover, decreasing the frequency of asking questions to users implies saving communication (e.g., bandwidth) and electronic resources (e.g., mobile phone battery) that are used for asking the questions, as the questions are usually asked using electronic communication devices, such as a mobile phone.

Therefore, a need exists for a method and apparatus for estimating the subjective wellbeing of a user in an accurate, personalized and resource saving manner, while at the same time minimizing the interaction with the user (e.g., minimizing the frequency of questioning the user) and enabling accurate recommendations for behavior modification to be generated that improve the user's wellbeing and avoid undesired behavior in the future.

### SUMMARY

A method and system for efficient estimation of a user's subjective wellbeing (SWB) score and for efficient generation of recommendations to improve the user's SWB score. The novel system generates the SWB score and recommendations using a range of continuously captured signals, including user inputs, body signals, signals captured by wearable devices, ambient sensors and environmental sensors. The system applies algorithmic modules to quantify factors describing the user's surroundings in order to generate the SWB score and to train a wellbeing management model that recommends behavioral modifications to improve the user's SWB score.

A method for improving a user's wellbeing uses a machine learning model to evaluate statistical parameters and to recommend ranges of those parameters that will improve the user's wellbeing score. Data related to the user is received onto an electronic device, including ambient data related to a location of the user, environmental data related to the location of the user, physiological data related to the user's body, and behavioral data of the user. In some embodiments, data is not used from each of the ambient, environmental, physiological and behavioral categories of data. Statistical parameters are generated that characterize the data by processing the data based on the period of time that corresponds to the data. A wellbeing score for the user is generated by applying a probabilistic model to the statistical parameters.

The user belongs to a group of similar users, and the model uses data from the similar users to generate its output. The probabilistic model is adjusted based on periodically received wellbeing information of all of the group of users that is directly received from each user of the group of users through a user interface of a communications device of each user of the group of users. The probabilistic model is also adjusted based on statistical parameters generated by processing data related to all of the group of users, including ambient data related to a location of each user of the group, environmental data related to the location of each user of the group, physiological data related to the body of each user of the group, and behavioral data of each user of the group. The model determines a recommended range of values for the parameters related to the ambient data, the environmental data, the physiological data and the behavioral data. The recommended range of parameter values is calculated to improve the wellbeing score of the user based on the statistical parameters and the wellbeing score. Behavior modifications are recommended to the user that will improve the user's wellbeing score based on the recommended range of values for the parameters.

In one embodiment, the generating of the wellbeing score involves generating a semi-static wellbeing score and a dynamic wellbeing score. The semi-static wellbeing score is generated by applying a first probabilistic model to the statistical parameters that characterize a statistical distribution of the data related to the user based on the period of time that corresponds to the data. The dynamic wellbeing score is generated by applying a second probabilistic model to a vector of percentile values that characterizes the data related to the user based on the period of time that corresponds to the data.

An electronic system for improving a user's wellbeing includes a receiver and a processor of a first electronic device and a receiver and user interface of a second electronic device. The receiver of the first electronic device is configured to receive, through a communications network, data related to the user that includes ambient data and environmental data related to the user's location, physiological data related to the user's body, and behavioral data of the user. The processor of the first electronic device is configured to generate statistical parameters that characterize the data related to the user. The statistical parameters are generated by processing the data based on the period of time that corresponds to the data. A wellbeing score for the user is generated by applying a probabilistic model to the statistical parameters.

The user belongs to a group of similarly situated users. The processor of the first electronic device is configured to adjust the probabilistic model based on periodically received wellbeing information from all of the users of the group that is directly received from each user of the group through a communications device of each user. A recommended range of values is determined for the parameters related to the ambient, environmental, physiological and behavioral data that will improve the wellbeing score of the user based on the statistical parameters and the wellbeing score. Behavior modifications are recommended to the user that will improve the wellbeing score of the user based on the recommended range of values for the parameters. A receiver of a second electronic device is configured to receive the recommended behavior modifications, and a user interface of the second electronic device is configured to display the recommended behavior modifications to the user.

Other embodiments and advantages are described in the detailed description below. This summary does not purport to define the invention. The invention is defined by the claims.

### BRIEF DESCRIPTION OF THE DRAWING

The accompanying drawings, where like numerals indicate like components, illustrate embodiments of the invention.

FIG. 1 shows a schematic graphic of the extraction of longitudinally monitored indexes according to an embodiment of the present invention.

FIG. 2 shows a schematic graphic of the quantification of dynamic values of longitudinally monitored indexes according to an embodiment of the present invention.

FIG. 3 shows a block data flow of the development of semi-static subjective wellbeing (SWB) models according to an embodiment of the invention.

FIG. 4 shows a block data flow of the development of dynamic SWB models according to an embodiment of the invention.

FIG. 5 shows a block data flow of the development of the wellbeing management model according to an embodiment of the invention.

### DETAILED DESCRIPTION

Reference will now be made in detail to some embodiments of the invention, examples of which are illustrated in the accompanying drawings.

A method and system are disclosed for efficiently measuring and estimating a user's subjective wellbeing (SWB) score, and for managing wellbeing and making wellbeing recommendations. The method performs user data collection, data analysis, SWB score modeling and generation of wellbeing recommendations. The system enables the user to understand and model the link between SWB and behavioral and surrounding factors. Using the method, the user's wellbeing will ultimately be understood. The method builds a wellbeing management model (WBMM) based on the analysis of a set of dynamic and static parameters describing an individual's behavior, states and characteristics, which are sensed automatically or reported by the user. The WBMM is developed by using wellbeing indexes and activities, behaviors, states and characteristics or a sub-set of these. The WBMM is also generated using the data about activities, behavior, states and similar characteristics derived from data relating to other users. The method also delivers feedback and/or recommendations to the user (for example, through a user interface) for improving the user's wellbeing. The feedback is delivered in the form of text, speech, images or video.

In a first aspect, a computer implemented method efficiently estimates and improves the subjective wellbeing state of a user who belongs to a group of users (persons). The method is implemented using a first electronic device. a) In step a), the first electronic device receives data through a communications network, for example, in the form of continuous signals. The data relates to the user and is acquired by one or more electronic devices. Examples of the data include ambient data related to a location of the user, environmental data related to the location of the user, physiological data of the user's body and behavioral data of the user. b) In step b), the first electronic device obtains statistical parameters characterizing the acquired data related to the user. For example, the parameters characterize the statistical distribution information of the data related to the user during a certain period of time and percentile information dynamically observed in the user's data. The statistical parameters are extracted by processing the data related to the user received in step a) according to a certain period of time. c) In step c), the first electronic device applies at least one probabilistic model to the extracted statistical parameters in order to obtain an estimated wellbeing score for the user. In one embodiment, two models are applied: one model to estimate the semi-static component of the wellbeing, and another model to estimate the dynamic component of the wellbeing. d) In step d), artificial intelligence is used to determine a recommended range of values for parameters related to the user based at least on the statistical parameters obtained in step b) and on the estimated wellbeing score of the user obtained in step c). The artificial intelligence techniques are performed by an artificial intelligent module of the system. The recommended range of values typically results in an improvement of the wellbeing score of the user (and for this reason they are recommended to the user). The determining of step d) is typically performed on the first electronic device, but may alternatively be performed in a different electronic device.

In one embodiment, the probabilistic models applied in step c) are generated and adjusted based on periodically received wellbeing information of all the users of the group. The wellbeing information is received through a communications network, such as using the first communications device. The wellbeing information is directly obtained from each user of the group through a user interface of a communications device of each user.

The following steps are performed in order directly to obtain wellbeing information of all of the users of the group.

A questionnaire is periodically presented to each user of the group through the user interface of the communications device of each user. The questionnaire was previously received onto the communications device of each user. Answers to the questionnaire from each user of the group are received through the user interface of the communications device of each user. In one embodiment, the answers containing wellbeing information are sent to the system by each communications device of each user in order to adjust the probabilistic models. The questionnaire is presented to the user so that the system can directly obtain wellbeing information within a certain time period (a year, month, week, . . . ) that is longer than the period on which user's related data is received in step a). In other words, the wellbeing information directly obtained from the users is obtained with much less frequency than the other types of user's related data.

In another embodiment, data related to all of the users of the group, which is captured using electronic devices, is used to generate the models. Examples of the type of data include ambient data related to a location of the users, environmental data related to the location of the users, physiological data of the users' bodies and/or behavioral data of the users. In another embodiment, parameters characterizing the statistical distribution information of all the users' (of the group) data are used to generate the semi-static wellbeing component model. In addition, percentile information dynamically observed in the data of all users of the group is used to generate the dynamic wellbeing component model.

In one embodiment, personal characteristics of all of the users of the group, which is captured using electronic devices, is used to generate the models. Examples of the communications devices of the users of the group include a laptop, a tablet, a personal computer, a portable computer, a mobile phone, a smart phone or any other electronic communications device.

In one embodiment, the statistical parameters extracted in step b) are of at least two types: parameters characterizing the statistical distribution of the data related to the user during the predetermined period of time and at least one vector of percentile values dynamically characterizing the data related to the user. Typically a shorter period of time is used for the percentiles (e.g., a day, half a day) than that used for the statistical distribution (e.g., a week).

A semi-static wellbeing score and a dynamic subjective well being score are estimated. The estimated semi-static wellbeing is obtained by applying a first probabilistic model (called the semi-static wellbeing component model) to the parameters that characterize the statistical distribution of the data related to the user during the certain period of time. Then the estimated dynamic wellbeing is obtained by applying a second probabilistic model (called the dynamic wellbeing component model) to one or more vectors of percentile values that dynamically characterize the data related to the user.

In another embodiment, the first electronic device is a remote server, and the electronic devices that acquire the data related to the users send the acquired data to the server through a communications network. The first electronic device is an user's communications device, and the one or more electronic devices that acquire the data related to the users (which are different than the user's communications device) send the acquired data through the communications network to the user's communications device. The acquired data belongs to four types of data: ambient data related to a location of the user, environmental data related to the location of the user, physiological data of the user's body, and behavioral data of the user. The data is acquired and received by the first electronic device in step a) and then used in the remaining steps. In other embodiments, only data belonging to one, two or three of these types of data is used.

Optionally, the recommended range of values for parameters that are determined for the user in step d) is sent by the first electronic device to the user's communication device through the communications network.

Personal characteristics of the user are also taken into account in step c) by the probabilistic models in order to obtain the estimated wellbeing score of the user and/or are taken into account in step d) to determine the recommended range of values for parameters related to the user. The personal characteristics used in step c) include static or semi-static attributes assigned to a user relating to a user segment, personal traits, self-esteem, tolerance of uncertainty or other types of personality characteristics. The personal characteristics can be obtained using known mechanisms, such as by inferring from the direct input from the user using questionnaires and surveys, by defining scales and inventories, and by applying pre-defined models to the user's data obtained from sensors.

Examples of the acquired environmental data includes parameters relating to pollution, outdoor light level, light level, weather, humidity, outdoor temperature (or any other environmental factor affecting the user). Examples of the behavioral data includes parameters relating to mobility, social interactions, sleep state, or any other behavioral parameter of the user. Examples of the physiological data includes parameters relating to the user's body, such as galvanic skin response, heart rate variability, skin temperature, or any measurement of any physical parameter of the user's body. Examples of the ambient data are includes parameters relating to indoor temperature, indoor light level, light exposure, noise level or any other ambient parameter affecting the user. Examples of the electronic devices that acquire data signals related to the user include usage sensors or logs embedded in a communications device of the user, wearable electronic devices with sensors to measure body signals, presence sensors, location sensors, smart environment devices, bed sensors, environment sensors to monitor environmental parameters such as temperature, air quality and weather information.

The novel electronic system for efficiently estimating and improving the wellbeing state of a user of a group of users includes a communication module and a processor. The communication module (or other communication means) receives, through a communications network, data related to the user that is acquired by one or more electronic devices. Examples of the data include ambient data related to the location of the user, environmental data related to the location of the user, physiological data of the user's body and behavioral data of the user. The processor is configured to obtain statistical parameters, to apply a probabilistic model to parameters, and to determine a recommended range of values for the parameters related to the user. The processor obtains statistical parameters that characterize the acquired data related to the user. The statistical parameters are extracted by processing the data related to the user that is associated with a predetermined period of time. The processor applies at least one probabilistic model to the extracted statistical parameters so as to obtain an estimated wellbeing score for the user. The processor determines, by using artificial intelligence, a recommended range of values for parameters related to the user based on the extracted statistical parameters that characterize the data related to the user and then estimates a wellbeing score of the user. Artificial intelligence is used to recommended range of values for parameters related to the user that will result in an improvement of the wellbeing score of the user.

The communications module is configured to receive through the communications network the wellbeing information of all of the users of the group, which is directly obtained from each user of the group through a user interface of a communications device of each user. The wellbeing information includes data related to all the users of the group and personal characteristics of all the users of the group. The processor generates at least one probabilistic model and adjusts the model based on the received wellbeing information of all the users of the group and on the personal characteristics of all of the users of the group.

In another embodiment, a computer program product includes computer program code adapted to perform the novel method. The program code is executed on a computer, a digital signal processor, a field-programmable gate array, an application-specific integrated circuit, a micro processor, a microcontroller, or any other form of programmable hardware. A non-transitory digital data storage medium stores the computer program that includes the instructions that cause the computer that executes the program to perform the novel method.

A method and system are disclosed for efficiently measuring and estimating the subjective wellbeing (SWB) score of a user (a person) and for efficiently generating recommendations for the user to improve the user's SWB score. To improve the user's SWB score, the method and system use a range of continuously captured signals (user inputs, body signals, signals captured by wearable devices, ambient and/or environmental sensors) and apply a novel mechanism to quantify the user's phenotype and surrounding factors in order to generate a subjective wellbeing (SWB) score and to generate a wellbeing management model that aims to improve SWB score.

The novel method provides a new way of inferring SWB in a passive way by creating a model that maps behavioral, body, ambient and environmental signals to SWB through analysis of the distribution of these signals to model the semi-static component of SWB (e.g., reflective wellbeing) and through calculating percentiles (for a shorter time frame) of these distributions for modeling the dynamic component of SWB (e.g., experiential wellbeing).

In prior art methods for evaluating wellbeing, the user is frequently asked to evaluate his wellbeing, such as the user's life-satisfaction and experiential wellbeing. However, frequently asking the user about his wellbeing has several drawbacks, including a higher expenditure of resources. In order to avoid these drawbacks, the novel method and system determine the SWB score in a passive way by creating a model based on behavioral, body, ambient and environmental information. In the embodiments, the frequency of asking the user for self evaluation is significantly reduced compared to the prior art methods because the user is only directly questioned using an electronic communications device in order to obtain ground-truth information and occasionally to calibrate the novel system. The term “ground-truth” refers to the process of gathering the proper objective data for the learning process of the system. The “ground-truth” is the information provided by direct observation (empirical evidence) as opposed to information provided by inference.

Hence, in the novel system and method SWB is inferred in a passive way in order to generate a wellbeing management model by inferring the main drivers of SWB. This not only avoids the drawbacks of the prior art, but it also enables both users and medical practitioners to better understand the positive and negative consequences of specific behavior, the intertwined effects of that behavior, and the surrounding factors.

The novel system monitors and measures several parameters (called factors) that depend on or affect the user's subjective wellbeing. Examples of these parameters include environmental factors (such as pollution, outdoor light level, light level, weather, humidity, outdoor temperature or any other environmental factor), sensed behavior (such as mobility, social interactions, sleep state), body signals (such as galvanic skin response, heart rate variability, skin temperature or the measurement of any physical parameter of the user's body), and ambient parameters (such as indoor temperature, indoor light level, light exposure, noise level and any other ambient parameter). Other types of parameters can also be monitored and used to generate the wellbeing management model and to estimate the SWB score.

In one embodiment, the method uses all four types of parameters (environmental, sensed behaviors, body signals or ambient parameters). In another embodiments, the method uses only one or several of the types of parameters. Generally speaking, the more types and number of parameters used, the more accurate the estimation of the SWB score will be; however, more resources will also be spent.

The system may also use information captured directly from users through questionnaires and surveys about their traits, demographics and SWB measurements. However, the types of parameters obtained by directly asking the user are obtained with much less frequency than are the other types of parameters. Parameters are asked directly of the user only to obtain ground-truth information and to calibrate the system.

The system includes at least four components: the user's communications device, usage logs and embedded sensors, dedicated body sensors, and indoor and outdoor sensors.

User's communications device. The parameters that are monitored and used by the system are captured from one or more sources of data, such as continuous signals. The system uses one or more electronic devices to obtain the values of the parameters. In order to ask questions of the user and to receive direct user inputs, the system uses the user's communications device. For example, the user responds to a survey or a questionnaire and answers the questions asked of the user on the user's communications device. Examples of the users' communications device include a laptop, a tablet, a personal computer, a portable computer, a mobile phone, a smart phone, any personal communications device or any electronic communications device. The user's communications device must have an electronic user interface, such as a screen, a keyboard, a speaker and a microphone in order for the system to asks questions of the user and to receive the user's answers.

Usage logs and embedded sensors. Personal devices such as wearable devices, hand-held devices, mobile phones, smartphones, tablets and laptops include embedded sensors and generate usage logs. The personal device may be the user's communications device employed to ask questions and to receive inputs from the user or from a different device. The user's communications device, such as a mobile phone, can also be used to monitor other parameters besides direct user inputs. These parameters include body signals of the user such as heart rate, ambient parameters such as light level, environmental factors, and behavior parameters such as social interactions.

Dedicated body sensors. The body sensors measure body signals and may be included in wearable electronic devices, such as smart-watches, bracelets (e.g., Fitbit, Jawbone), clips (e.g., Fitbit) or any other type of wearable electronic devices that can be worn by the user.

Indoor and outdoor sensors. Ambient (indoor) sensors include presence sensors, location sensors, smart environments devices and bed sensors. Environment (outdoor) sensors monitor environmental parameters, such as temperature, air quality, weather information and any other environment parameters.

Some data used by the system is obtained from external sources such as dedicated databases, news outlets and social networks.

Any known electronic device that can be used to sense or measure the values of the parameters can be used by the system. A single electronic device can be used to obtain the value of different types of parameters. For example, a wearable device can be used to obtain body signals as well as environmental factors. And a mobile phone can be used to obtain direct inputs from the user as well as body signals, ambient parameters, environmental factors and behavioral parameters.

The data captured for a specific user over a period of time is called longitudinal data and is stored in a user database. All the parameter values obtained by the various electronic devices are sent by those electronic devices to the database. The database can be internal to one of the electronic devices, such as the user's communications device, or can be external to all of the electronic devices used to obtain the data. Such an external database can be a remote server. The electronic device used to obtain the longitudinal data should have a communications interface that allows the electronic device to transmit the obtained data to the database over the communications network either directly or through another electronic device. The communications network can be a wireless communications network, a mobile communications network such as GSM 2G, GPRS, UMTS, 3G, 4G or LTE, LAN or W-LAN or any other type of wired or wireless communication networks.

For simplicity, all captured data can be stored in a single database. It is possible, however, to store the data in multiple databases in the same or different electronic devices.

In the novel method, the data is not used in its original form as captured by the various data sources and as the user inputs the data into the user's communications device, hand-held device, wearable device, ambient sensor, or environmental monitor. Instead, the data is first processed using computer based techniques to obtain better quantifiable, usable and representative data, which is called indexes or indices. In order to process the data as originally captured, the data that is entered into the user database is fed into various modules to obtain a set of indexes. These modules are functional blocks implemented by a computer processor that apply the method's models to obtain the indexes. The particular model that is applied (whether externally developed or pre-defined) depends on the type of parameters being processed. For example, a behavior recognition model is applied to the behavioral parameters from the database in order to obtain behavioral indexes. A physiological state model is applied to the body parameters from the database in order to obtain body (physiological) indexes. An ambient state model is applied to the ambient parameters from the database in order to obtain ambient indexes. And an environmental state model is applied to the environmental parameters from the database in order to obtain environmental indexes. These are non-limiting examples, and other types of parameters and models can also be used.

In one embodiment, all of the four types of parameters are captured, and consequently all of the models are used to process those parameters. In other embodiments, only one or a couple of these types of parameters are used, so only some of the models are applied.

Any of the models can be used to obtain usable, quantifiable and representative indexes of user information from the data in the user database. For instance, in one model behavior is defined as a pattern of activities of daily living, such as eating, sleeping, doing sports, socializing, etc. The novel system infers activities or behavior of the user based either on the direct input from the user or on the output from the electronic devices and sensors of the system. The system uses pre-defined models to convert the signals and raw data into behavior patterns. Then from the behavior patterns, the behavioral indexes are derived. In the case of physiological data, physiological states represent dynamically changing information about the user (such as heart rate variability or galvanic skin response) that often is linked to an emotional state or mood. The system infers the states based on the direct input from the user or on the output from any of the electronic devices and sensors. The system uses the pre-defined models of converting the signals into physiological states.

Personal characteristics include static and semi-static (slowly changing) attributes assigned to a user and are associated with a user segment, demographics, personal traits (e.g., proneness to boredom), self-esteem, tolerance of uncertainty, or any other type of personality characteristics. The personal characteristics are inferred from the direct input from the user using questionnaires, surveys, defined scales, and inventories or can be determined by applying models to data acquired from sensors. The system uses the predetermined models to convert the raw data signals into personal characteristics and produces scores that then represent the attributes associated with the specific user. Typically, the personal characteristics are more static (i.e. less variable) than the environmental, behavioral, body and ambient parameters.

The user data (both raw data stored in the database or derived indexes) is further processed by a computer processor in order to generate the specific input for the SWB models. More specifically, this further processing is performed to obtain a semi-static representation (or component) of the user data and a dynamic representation (or component) of the user data. The semi-static representation of the user data is based on the characterization of the distribution of data. The dynamic representation of the user data is a current value represented as a percentile of the distribution of the data.

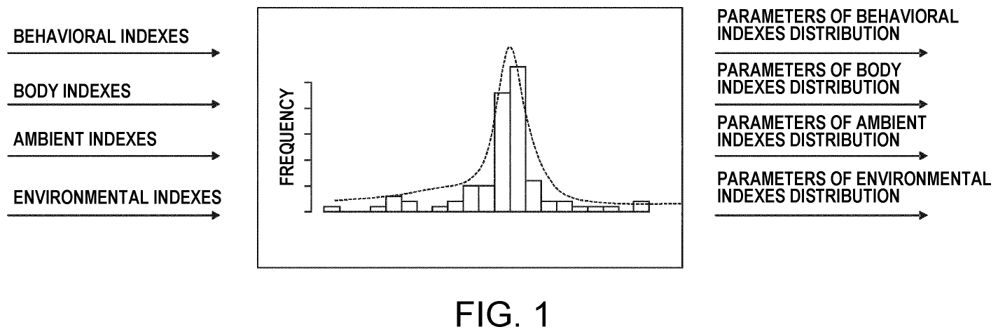

As part of the novel method, the user behavioral, body, ambient and environmental data in the form of indexes is processed in order to extract the statistical distribution of the indexes. FIG. 1 illustrates the statistical distribution of the indexes.

In order to model the semi-static parameters of the user's phenotype and the surrounding factors, each of the indexes (each type of data) is analyzed according to its distribution of values. Each of the indexes is analyzed by the distribution of the frequency of each value collected longitudinally over a certain period of time, such as a week, a month, or a year.

Analyzing the distribution of the frequency of values involves extracting the distribution of parameters so that each index (behavioral, body, ambient, environmental) is converted into a set of statistical distribution parameters. For instance, if it is a near-normal distribution, these distribution parameters will include mean and standard deviation. For a power law distribution, the parameters will include Xmin and α, followed by the extraction of Kolmogorov-Smirnov coefficients about the distribution fit. The goal is to quantify typical patterns of a specific index per user, and quantify baseline behaviors and surrounding factors. These semi-static parameters are used to model the semi-static SWB component.

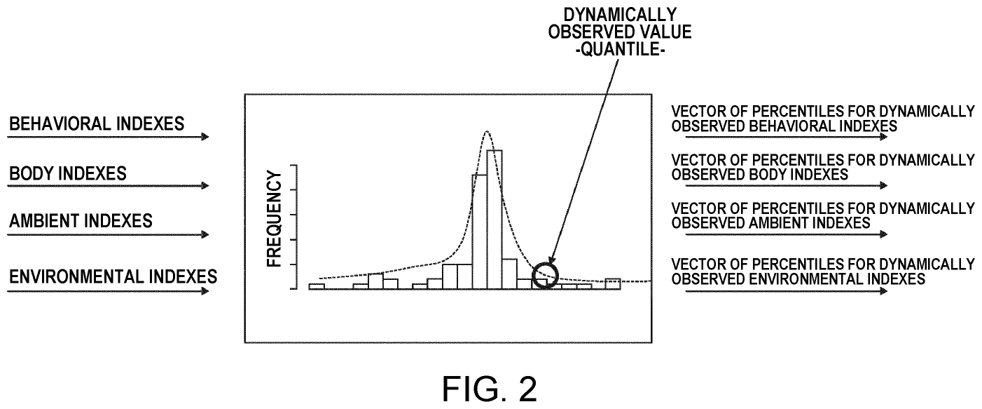

The novel method also quantifies the dynamic behavioral and surrounding parameters. In order to do so, considering the range of longitudinal values (i.e., the distribution), dynamic values that are observed during a specific time period (such as, part of a day, a day, weekend, week, or month) are delivered to the model to obtain an estimation of the dynamic SWB. The values used by the model are not absolute values, but rather percentiles of the respective distributions, such as the distribution of frequency of each value as shown in FIG. 2. For example, consider a situation in which a user makes 8000 steps a day, with an average speed of 6 km/h, and over time both speed and number of steps at a daily level follow a normal distribution during a period of the user's life in which the system has been monitoring the user. If today the user made 1000 steps with a speed of 8 km/h, these dynamic values can be expressed as a percentile of the user's typical distribution to indicate that the user's current number of steps belongs to the 25th percentile (lower than 75% of the user's days) but the user's speed belongs to 90th percentile (in top 10% of the user's days).

The novel method uses percentiles instead of absolute values in order to extract features (i.e., parameters) that are used as inputs into the models. The method avoids the disadvantages of other models that use absolute values as input parameters, such as daily mean of heart rate, sleep hours, daily time at home or work, daily time with friends, daily ambient temperature, etc. The absolute values of user data do not capture individual differences well. For example, a value that is an outlier for user 1 may be a mean value for user 2. The method more accurately recognizes a differentiation between users using less captured data by using percentiles instead of absolute values. FIG. 2 illustrates this part of the method in which the user behavioral, body, ambient and environmental data in the form of indexes is processed to extract the percentiles of the distributions of each captured parameter.

In one embodiment, a vector of percentiles is extracted for each type of parameters (behavioral, body, ambient, environmental). The dimension of the vector depends on how many parameters (variables) are monitored for each of the data types. For example, if the monitored environmental parameters are pollution level, temperature and humidity, the vector of percentiles of the environmental parameters will have a dimension equal to three. The exact percentile values depends on the distribution of the parameter values observed over a certain period of time. For example, considering again the example above for steps and walking speed, if the user's wearable (e.g., Fitbit) observes that today the user has made 1000 steps, which belongs to the 25th percentile with respect to the distribution of daily steps in the user's life, this 25th percentile will be an observed value for today. In addition, the value of 25 may be one element of a behavioral vector. Other elements can be the percentile of the number of hours that the user has slept, and the number of locations that the user has visited today, etc. In the same manner, humidity level and outdoor temperature can be two elements of ambient vector, also expressed as percentiles depending on the distribution of these parameters at the location where the user lives.

In one embodiment, using the interactive communication module of the user's mobile phone, the system periodically captures semi-static and dynamic SWB measurements. For example, the system captures semi-static SWB data by posing questions to the user, such as “Overall, how satisfied are you with your life nowadays?” and “Overall, to what extent do you feel the things you do in your life are worthwhile?” For example, the system captures dynamic SWB data by posing questions to the user, such as “How happy did you feel?” and “How worthwhile did this feel?” in reference to a specific current activity. The answers to these questions are used as ground-truth information for developing and calibrating the probabilistic model of the system. These questions are only posed from time to time in order to develop the model and for calibration and not for updating the user's SWB in real time on an ongoing basis. Because the behavior recommendations to the user are generated based on the recommended range of values for parameters that are related to ambient, environmental, physiological and behavioral data acquired from sensors, the need to have frequent self-reports from the user in order to monitor SWB and understand its drivers is reduced.

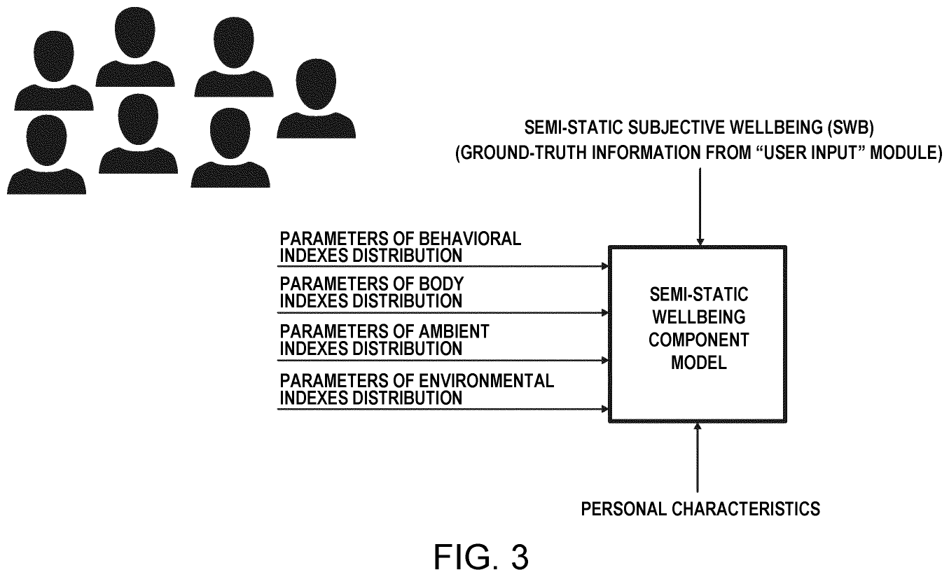

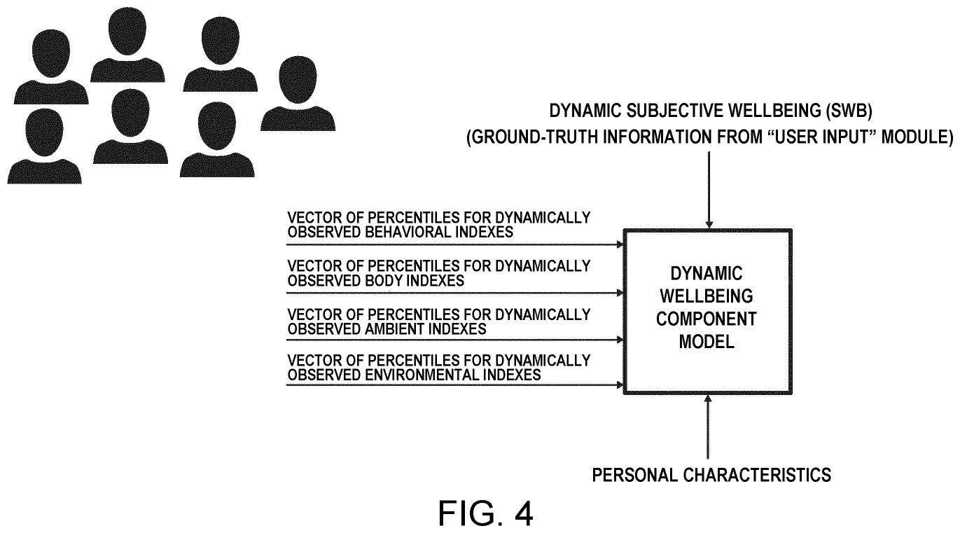

FIGS. 3 and 4 illustrate the inputs to the semi-static and dynamic SWB models using the database of users who use the novel system to estimate their subjective wellbeing (SWB). FIG. 3 illustrates how a computer processor generates a probabilistic model of semi-static SWB by mapping longitudinal characterizations of behavioral, body, ambient and/or environmental parameters of the users whose data is stored in the database to semi-static SWB self-reported by the users whose data is stored in the database. Optionally, the personal characteristics of the users whose data is stored in the database are also mapped to the self-reported, semi-static SWB. The longitudinal characterizations of behavioral, body, ambient and/or environmental parameters are the extracted distribution parameters output in FIG. 1. The probabilistic model is also called the “Semi-Static Wellbeing Component Model”. Semi-static SWB is sometimes referred to as “life-satisfaction”.

The model that determines the semi-static SWB component of a user's SWB is designed by using the statistical distribution parameters of the monitored data of the users (behavioral, body, ambient and/or environmental data). Optionally, the model is adjusted and calibrated by using as ground truth data the personal characteristic of the users as well as the self-reported, semi-static SWB. The “Semi-Static Wellbeing Component Model” is generated, adjusted and calibrated using machine learning (generally called artificial intelligence) on the available data.

In one embodiment, the SWB models are created using supervised machine-learning classification algorithms, such as a Bayesian network algorithm, a linear regression algorithm, a support vector machine algorithm, a decision tree algorithm (e.g., XGBOOST or Random Forests), a hidden Markov model algorithm or any other algorithm. The SWB model can be tuned to balance precision. FIG. 4 illustrates the mapping of vectors of percentiles for dynamically observed behavioral, body, ambient and environmental values of the users together with personal characteristics of the users to dynamic SWB that is self-reported by the users. The mapping of the vectors and personal characteristics to the dynamic SWB generates the probabilistic model for dynamic SWB. The model for dynamic SWB (i.e., experiential wellbeing) is also called the Dynamic Wellbeing Component Model. The model to estimate the dynamic SWB component of a user's SWB maps the percentiles of the monitored data of the users (behavioral, body, ambient and/or environmental) and the personal characteristic of the users to the dynamic self-reported SWB ground-truth data. This mapping is also used to adjust and calibrate the model by training the model using machine-learning algorithms.

The semi-static wellbeing component and the dynamic wellbeing component of the SWB model are generated using information from all of the users whose data is stored in the database or at least a significant group of those users. Thus, the model is generated using information from a large group of users as opposed to from a specific user. In order to estimate the semi-static SWB component score and the dynamic SWB component score of a specific user, each model component is queried with the data of the specific user. Distribution parameters of ambient, environmental, body, and behavioral data (and optionally also personal characteristics) of the user are the input to the semi-static SWB component model to generate the estimated semi-static SWB score for the specific user. Vectors of percentiles of ambient, environmental, body and behavioral data (and optionally also personal characteristics) of the user are the input to the dynamic SWB component model to generate the estimated dynamic SWB score for the specific user.

The group of users whose data is used to build the semi-static SWB model and the dynamic SWB model can be all of the users whose data is stored in the database. These users are a group of users to which the user is classified or with which the user has something in common. For example, the users of the group can be users of the same wellbeing service, users for whom their wellbeing is also being estimated, users from the same location (city or neighborhood) as the user, or users with the same professional activity as the user. By using data from all of the users in the database (or a significant group of them), a semi-static model and a dynamic model are developed that map all of the input variables (or a significant part of them) to wellbeing. For a new user, when the system has only partial data, such as one of the ambient, environmental, body or behavioral input variables which are all passively collected, the model can infer the semi-static SWB or dynamic SWB of the user without the need to question the user for direct input.

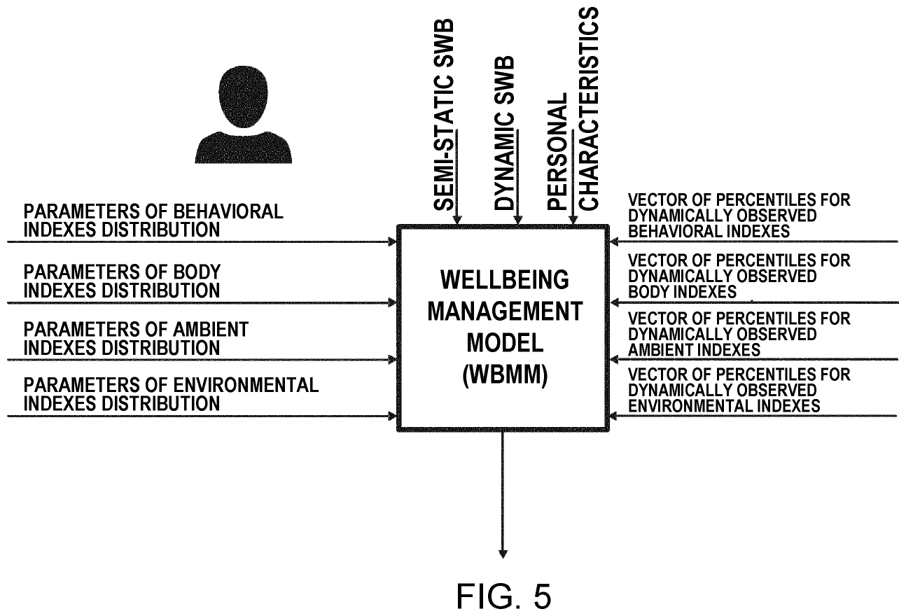

FIG. 5 shows the Wellbeing Management Model (WBMM) of the novel system. The WBMM is a functional block of the system implemented by a computer processor. The WBMM is an artificial intelligence driven module that uses data processed by the other models or obtained directly from the user as inputs to generate a set of recommendations for improving the user's subjective wellbeing (SWB). As shown in FIG. 5, the input to the wellbeing management model WBMM includes the distribution parameters of the user's behavioral, body, ambient and/or environmental data previously extracted, the vectors of percentiles extracted for dynamically observed behavioral, body, ambient and/or environmental data previously extracted, the semi-static SWB component score estimated for the user by the Semi-Static Wellbeing Component Model, the dynamic SWB component score estimated for the user by the Dynamic Wellbeing Component Model and optionally also the personal characteristics of the user. The WBMM uses these inputs to generate a recommended range of values for the behavioral, body, ambient and environmental parameters that will improve the SWB of the user. That is, this module determines a range of values that the behavioral, body, ambient and environmental parameters of the user must have in order to achieve a better subjective wellbeing.

Using the statistical distribution parameters (left hand side inputs in FIG. 5), the WBMM uses machine learning to learn the typical patterns of behavioral, physiological and other parameters surrounding the user in order to identify how variations in the parameters over the various time periods (percentile measures of dynamically observed indexes) impact the SWB that is determined in the semi-static and dynamic SWB component models. The WBMM of FIG. 5 relies on the semi-static and dynamic SWB models that operate on the whole dataset. However, the recommendations output by the WBMM, although based on similar inputs to the SWB models, are directed to a specific individual as opposed to a group. The recommendations are a range of values of behavioral, body, ambient and environmental parameters that in their combination as applied to the specific user result in an improved SWB. In order to derive personalized recommendations, the novel method performs a grid search by iteratively changing behavioral parameters beginning with smaller and progressing to larger changes until the positive impact on wellbeing of the changes peaks. For example, the WBMM block of the system uses a machine-learning model that is trained with the available data.

Although the figures show all of the types of parameters (sensed behaviors, body signals, ambient parameters, and environmental data) used as inputs for the models, use of all four types of parameters is not essential. In one embodiment, all four types of parameters are used (ambient, environmental, physiological and behavioral data). In other embodiments, only one or a couple of these parameters are used. The more types of parameters and amount of data used, the more accurate the estimation of the SWB score will be, but also the more resources will be spent.

Depending on the embodiment, the various modules of the system are in the same or in different physical locations (and different devices). In one embodiment, all modules are implemented in the same device (for example, a central or remote server). In other embodiments, the modules reside on multiple servers or on the mobile communications device. In other embodiments, some modules may be in the user's communications device and the rest in one or more servers. In the case that the modules are implemented on a server, the communications device communicates with the server through a communication networks, such as a wireless communications network, a mobile communications network as 2G, 3G, 4G or LTE, LAN or W-LAN, or any other type of communications networks.

Embodiments of the invention can be implemented on a variety of architectural platforms, operating and server systems, devices, systems, or applications. Any particular architectural layout or implementation presented herein is provided for purposes of illustration and comprehension only and does not limit the invention.

All statements herein reciting principles, aspects, and embodiments of the invention, as well as specific examples thereof, are intended to encompass equivalents thereof. Although the present invention has been described with reference to specific embodiments, it should be understood by those skilled in the art that the foregoing and various other changes, omissions and additions in the form and detail thereof may be made therein without departing from the scope of the invention as defined by the claims.

1.-15. (canceled)

16. A method for improving a user's wellbeing, comprising:
(a) receiving data related to the user onto a first electronic device, wherein the data includes ambient data related to a location of the user, environmental data related to the location of the user, physiological data related to the user's body, and behavioral data of the user;
(b) generating statistical parameters that characterize the data received in (a), wherein the statistical parameters are generated by processing the data based on a period of time that corresponds to the data;
(c) generating a wellbeing score for the user by applying a probabilistic model to the statistical parameters;
(d) determining a recommended range of values for parameters related to the ambient data, the environmental data, the physiological data and the behavioral data that will improve the wellbeing score of the user based on the statistical parameters generated in (b) and the wellbeing score generated in (c); and
(e) recommending behavior modifications to the user that will improve the wellbeing score of the user based on the recommended range of values for parameters determined in (d).

17. The method of claim 16, wherein the user belongs to a group of users, further comprising:
adjusting the probabilistic model based on periodically received wellbeing information of all of the group of users that is directly received from each user of the group of users through a user interface of a communications device of each user of the group of users.

18. The method of claim 16, wherein the user belongs to a group of users, further comprising:
adjusting the probabilistic model based on statistical parameters generated by processing data related to all of the group of users, wherein the data related to all of the group of users includes ambient data related to a location of each user of the group of users, environmental data related to the location of each user of the group of users, physiological data related to the body of each user of the group of users, and behavioral data of each user of the group of users.

19. The method of claim 17, wherein directly receiving wellbeing information from each user of the group of users further comprises:
periodically presenting a questionnaire to each user of the group of users through a user interface of the communications device of each user of the group of users; and
receiving a response to the questionnaire from each user of the group of users through the user interface of the communications device of each user of the group of users.

20. The method of claim 17, wherein the communications device of each user of the group of users is selected from the group consisting of: a laptop, a tablet, a personal computer, a portable computer, a mobile phone, and a smartphone.

21. The method of claim 16, wherein the statistical parameters are selected from the group consisting of: parameters that characterize a statistical distribution of the data related to the user based on the period of time that corresponds to the data, and a vector of percentile values that characterizes the data related to the user based on the period of time that corresponds to the data.

22. The method of claim 16, wherein the generating the wellbeing score in (c) involves generating a semi-static wellbeing score and a dynamic wellbeing score, wherein the semi-static wellbeing score is generated by applying a first probabilistic model to the statistical parameters that characterize a statistical distribution of the data related to the user based on the period of time that corresponds to the data, and wherein the dynamic wellbeing score is generated by applying a second probabilistic model to a vector of percentile values that characterizes the data related to the user based on the period of time that corresponds to the data.

23. The method of claim 16, wherein the first electronic device is a remote server, wherein the data related to the user is acquired by a second electronic device, and wherein the second electronic device transmits the data through a communications network to the remote server.

24. The method of claim 16, wherein the recommended range of values for parameters is transmitted by the first electronic device to a communications device of the user through a communications network.

25. The method of claim 16, wherein the probabilistic model uses personal characteristics of the user to generate the wellbeing score for the user, and wherein the personal characteristics are selected from the group consisting of: the user's degree of self esteem, the user's tolerance for uncertainty, and the user's proneness to boredom.

26. The method of claim 16, wherein the ambient data relates to parameters selected from the group consisting of: indoor temperature, indoor light level, the user's exposure to light, and noise level, wherein the environmental data relates to parameters selected from the group consisting of: pollution, outdoor light level, weather, humidity, and outdoor temperature, wherein the physiological data relates to parameters of the user's body selected from the group consisting of: galvanic skin response, heart rate variability, and skin temperature, and wherein the behavioral data relates to parameters selected from the group consisting of: mobility, social interactions, and sleep state.

27. The method of claim 16, wherein the data related to the user is acquired by a second electronic device, and wherein the second electronic device is selected from the group consisting of: a communications device of the user with an embedded sensor, a wearable device with a sensor that measures body signals of the user, a communications device of the user with a location sensor, a communications device of the user that determines ambient temperature, a motion detector, and an air quality sensor.

28. An electronic system for improving a user's wellbeing, comprising:
a receiver of a first electronic device configured to receive, through a communications network, data related to the user that includes ambient data related to a location of the user, environmental data related to the location of the user, physiological data related to the user's body, and behavioral data of the user; and
a processor of the first electronic device configured to:
generate statistical parameters that characterize the data related to the user, wherein the statistical parameters are generated by processing the data based on a period of time that corresponds to the data;
generate a wellbeing score for the user by applying a probabilistic model to the statistical parameters;
determine a recommended range of values for parameters related to the ambient data, the environmental data, the physiological data and the behavioral data that will improve the wellbeing score of the user based on the statistical parameters and the wellbeing score; and
recommend behavior modifications to the user that will improve the wellbeing score of the user based on the recommended range of values for parameters.

29. The electronic system of claim 28, further comprising:
a receiver of a second electronic device configured to receive the recommended behavior modifications; and
a user interface of the second electronic device configured to display the recommended behavior modifications.

30. The electronic system of claim 28, wherein the user belongs to a group of users, and wherein the processor of the first electronic device is further configured to adjust the probabilistic model based on periodically received wellbeing information of all of the group of users that is directly received from each user of the group of users through a communications device of each user of the group of users.

31. A method for improving a user's wellbeing, comprising:
receiving data related to the user onto a first electronic device, wherein the data is acquired by a second electronic device, and wherein the data is selected from the group consisting of: ambient data related to a location of the user, environmental data related to the location of the user, physiological data related to the user's body, and behavioral data of the user;
generating statistical parameters that characterize the data, wherein the statistical parameters are generated by processing the data based on a period of time to which the data corresponds;
generating a wellbeing score for the user by applying a probabilistic model to the statistical parameters;
determining a recommended range of values for parameters related to the data that will improve the wellbeing score of the user based on the statistical parameters and the wellbeing score; and
recommending behavior modifications to the user that will improve the wellbeing score of the user based on the recommended range of values for parameters.

32. The method of claim 31, further comprising:
receiving the recommended behavior modifications onto a second electronic device; and
displaying the recommended behavior modifications to the user on a user interface of the second electronic device.

33. The method of claim 31, wherein the user belongs to a group of users, further comprising:
adjusting the probabilistic model based on periodically received wellbeing information of all of the group of users that is directly received from each user of the group of users through a communications device of each user of the group of users.

34. The method of claim 33, wherein the wellbeing information that is directly received from each user of the group of users is generated by periodically presenting a questionnaire to each user of the group of users through the communications device of each user of the group of users, and receiving a response to the questionnaire from each user of the group of users through the communications device of each user of the group of users.

35. The method of claim 31, wherein the user belongs to a group of users, further comprising:
adjusting the probabilistic model based on statistical parameters generated by processing data related to all of the group of users, wherein the data related to all of the group of users includes data is selected from the group consisting of: ambient data related to a location of each user of the group of users, environmental data related to the location of each user of the group of users, physiological data related to the body of each user of the group of users, and behavioral data of each user of the group of users.
